(19) United States
(12) Patent Application Publication
(10) Pub. No.: US 2025/0082226 A1
(43) Pub. Date: Mar. 13, 2025

(54) SUBJECT BODY INDEX ESTIMATION METHOD FOR MEDICAL IMAGING AND MEDICAL IMAGING SYSTEM

(71) Applicant: GE Precision Healthcare LLC, Waukesha, WI (US)

(72) Inventors: Xiaolan Liu, Beijing (CN); Qingyu Dai, Beijing (CN); Ting Ye, Beijing (CN); Zhongyu You, Beijing (CN); Wenyan Ma, Beijing (CN); Jun Zhang, Beijing (CN)

(21) Appl. No.: 18/829,080

(22) Filed: Sep. 9, 2024

(30) Foreign Application Priority Data
Sep. 12, 2023 (CN) 202311170878.5

(51) Int. Cl. A61B 5/107; A61B 5/00

(52) U.S. Cl. CPC A61B 5/1079; A61B 5/7264

(57) ABSTRACT

A subject body index estimation method for medical imaging is presented. The method includes: obtaining image data of a subject captured by an image capture apparatus; extracting a first feature vector and key point information at a predetermined position of the body of the subject from the image data; generating a second feature vector based on the key point information; and estimating at least one of the height and the weight of the subject based on the first feature vector and the second feature vector.

### CROSS REFERENCE TO RELATED APPLICATIONS

The present application claims priority and benefit of Chinese Patent Application No. 202311170878.5 filed on Sep. 12, 2023, which is incorporated herein by reference in its entirety.

### TECHNICAL FIELD

Examples of the present application relate to the technical field of medical apparatuses, and in particular relate to a subject body index estimation method for medical imaging and a medical imaging system.

### BACKGROUND

In clinical scanning, indexes such as the age, height, and weight of a subject need to be routinely recorded prior to a medical imaging examination. For any medical imaging mode, the height and weight of the subject are important parameters. For example, in a magnetic resonance imaging system, the height and weight parameters may be used for positioning, as well as assisting with evaluation of a radio frequency absorption rate (SAR) value, to prevent a tissue to be imaged from absorbing excessive radio frequency energy in a short period of time, and generating local burns or causing even greater safety issues. For example, for a computed tomography system, an appropriate tube current may be determined based on the height and the weight, to minimize a radiation dose and the like.

### SUMMARY

Examples of the present application provide a subject body index estimation method for medical imaging and a medical imaging system.

According to one aspect of the examples of the present application, a subject body index estimation method for medical imaging is provided. The method includes: obtaining image data of a subject captured by an image capture apparatus; extracting a first feature vector and key point information at a predetermined position of the body of the subject from the image data; generating a second feature vector based on the key point information; and estimating at least one of the height and the weight of the subject based on the first feature vector and the second feature vector.

According to one aspect of the examples of the present application, a computer-readable storage medium is provided. The computer-readable storage medium includes a stored computer program. The subject body index estimation method for medical imaging described in the foregoing aspect is performed when the computer program is run.

According to one aspect of the examples of the present application, a medical imaging system is provided. The system comprises: an image capture apparatus, capturing image data of a subject; and a controller, connected to the image capture apparatus and configured to perform the subject body index estimation method in the foregoing aspect.

One of the beneficial effects of the examples of the present application lies in: extracting a first feature vector and key point information at a predetermined position of the body of the subject from image data captured by an image capture apparatus; generating a second feature vector based on the key point information; and estimating at least one of the height and the weight of the subject by combining the first feature vector and the second feature vector. Therefore, the height and weight of a human body can be estimated by combining a global first feature in original image data and a second feature related to human body morphology, that is, the height and weight of a human body can be estimated using multi-dimensional features, so that the estimation result is more accurate.

With reference to the following description and drawings, specific embodiments of the examples of the present application are disclosed in detail, and the means by which the principles of the examples of the present application can be employed are illustrated. It should be understood that the embodiments of the present application are therefore not limited in scope. Within the scope of the spirit and clauses of the appended claims, the embodiments of the present application include many changes, modifications, and equivalents.

### BRIEF DESCRIPTION OF THE DRAWINGS

The included drawings are used to provide further understanding of the examples of the present application, which constitute a part of the description and are used to illustrate the embodiments of the present application and explain the principles of the present application together with textual description. Evidently, the drawings in the following description are merely some examples of the present application, and a person of ordinary skill in the art may obtain other embodiments according to the drawings without involving inventive skill. In the drawings:

FIG. 1 is a schematic diagram of a magnetic resonance imaging system according to an example of the present application;

FIG. 2 is a schematic diagram of a subject body index estimation method for medical imaging according to an example of the present application;

FIG. 3 is a schematic diagram of a medical imaging system according to an example of the present application;

FIG. 4 is a schematic diagram of a magnetic resonance imaging system according to an example of the present application;

FIG. 5 is a schematic diagram of a subject body index estimation apparatus for medical imaging according to an example of the present application;

FIG. 6 is a schematic diagram of a subject body index estimation method for medical imaging according to an example of the present application;

FIG. 7 is a schematic diagram of a subject body index estimation method for medical imaging according to an example of the present application;

FIG. 8 is a schematic diagram of a subject body index estimation apparatus for medical imaging according to an example of the present application;

FIG. 9 is a schematic diagram of a convolutional neural network according to an example of the present application; and

FIG. 10 is a schematic diagram of key points at predetermined positions according to an example of the present application.

### DETAILED DESCRIPTION

The foregoing and other features of the examples of the present application will become apparent from the following description and with reference to the drawings. In the description and drawings, specific embodiments of the present application are disclosed in detail, and part of the embodiments in which the principles of the examples of the present application may be employed are indicated. It should be understood that the present application is not limited to the described embodiments. On the contrary, the examples of the present application include all modifications, variations, and equivalents which fall within the scope of the appended claims.

In the examples of the present application, the terms “first” and “second” and so on are used to distinguish different elements from one another by their title, but do not represent the spatial arrangement, temporal order, or the like of the elements, and the elements should not be limited by said terms. The term “and/or” includes any one of and all combinations of one or more associated listed terms. The terms “comprise”, “include”, “have”, etc., refer to the presence of stated features, elements, components, or assemblies, but do not exclude the presence or addition of one or more other features, elements, components, or assemblies.

In the examples of the present application, the singular forms “a” and “the” or the like include plural forms, and should be broadly construed as “a type of” or “a kind of” rather than being limited to the meaning of “one”. Furthermore, the term “the” should be construed as including both the singular and plural forms, unless otherwise specified in the context. In addition, the term “according to” should be construed as “at least in part according to . . . ”, and the term “based on” should be construed as “at least in part based on . . . ”, unless otherwise clearly specified in the context.

The features described and/or illustrated for one embodiment may be used in one or more other embodiments in an identical or similar manner, combined with features in other embodiments, or replace features in other embodiments. The term “include/comprise” when used herein refers to the presence of features, integrated components, steps, or assemblies, but does not exclude the presence or addition of one or more other features, integrated components, steps, or assemblies.

A medical imaging system described herein includes, but is not limited to, a computed tomography (CT) system, a magnetic resonance imaging (MRI) system, a C-arm imaging system, a positron emission computed tomography (PET) system, a single photon emission computed tomography (SPECT) system, an ultrasonic system, an X-ray imaging system, or any other suitable medical imaging system.

In the following, a magnetic resonance imaging system is used as an example for description, but examples of the present application are not limited thereto.

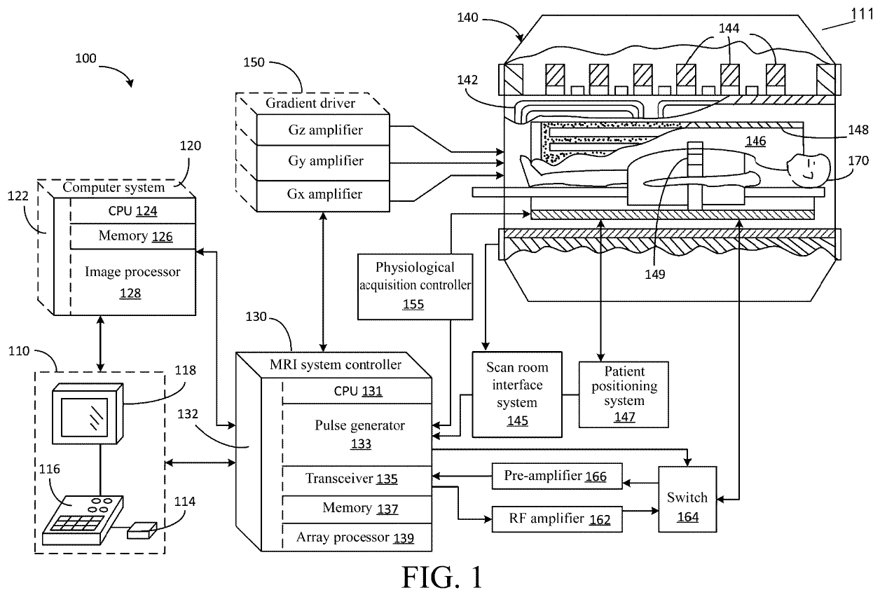

For ease of understanding, FIG. 1 shows a magnetic resonance imaging (MRI) system 100 according to some examples of the present invention.

The MRI system 100 includes a scanning unit 111. The scanning unit 111 is configured to perform a magnetic resonance scan on a subject (for example, a human body) 170 to generate a reconstructed image of a region of interest of the subject 170. The region of interest may be a predetermined anatomical site or anatomical tissue.

Operation of the MRI system 100 is controlled by an operator workstation 110, and the operator workstation 110 includes an input device 114, a control panel 116, and a display 118. The input device 114 may be a joystick, a keyboard, a mouse, a trackball, a touch-activated screen, voice control, or any similar or equivalent input device. The control panel 116 may include a keyboard, a touch-activated screen, voice control, a button, a slider, or any similar or equivalent control device. The operator workstation 110 is coupled to and communicates with a computer system 120, and the computer system enables an operator to control the generation and viewing of an image on the display 118. The computer system 120 includes a plurality of components that communicate with one another by means of an electrical and/or data connection module 122. The connection module 122 may employ a direct wired connection, an optical fiber connection, a wireless communication link, etc. The computer system 120 may include a central processing unit (CPU) 124, a memory 126, and an image processor 128. In some embodiments, the image processor 128 may be replaced with an image processing function implemented in the CPU 124. The computer system 120 may be connected to an archival media device, a persistent or backup memory, or a network. The computer system 120 may be coupled to and communicate with a separate MRI system controller 130.

The MRI system controller 130 includes a set of components that communicate with one another by means of an electrical and/or data connection module 132. The connection module 132 may employ a direct wired connection, a fiber optic connection, a wireless communication link, etc. The MRI system controller 130 may include a CPU 131, a sequential pulse generator 133 that communicates with the operator workstation 110, a transceiver (or an RF transceiver) 135, a memory 137, and an array processor 139. In some embodiments, the sequential pulse generator 133 may be integrated into a resonance assembly 140 of the scanning unit 111 of the MRI system 100. The MRI system controller 130 may receive a command from the operator workstation 110, and is coupled to the scanning unit 111, to indicate an MRI scan sequence that is to be performed during an MRI scan, so as to control the scanning unit 111 to execute the described magnetic resonance scan procedure. The MRI system controller 130 is further coupled to and communicates with a gradient driver system 150, and the gradient driver system is coupled to a gradient coil assembly 142 to generate a magnetic field gradient during the MRI scan.

The sequential pulse generator 133 may further receive data from a physiological acquisition controller 155, the physiological acquisition controller receives signals from a plurality of different sensors (for example, electrocardiogram (ECG) signals from electrodes attached to a patient), the sensors being connected to the subject or patient 170 undergoing the MRI scan. The sequential pulse generator 133 is coupled to and communicates with a scan room interface system 145, and the scan room interface system receives signals from various sensors associated with the state of the resonance assembly 140. The scan room interface system 145 is further coupled to and communicates with a patient positioning system 147, and the patient positioning system sends and receives signals to control the movement of a patient table to a desired position to perform the MRI scan.

The MRI system controller 130 provides gradient waveforms to the gradient driver system 150, and the gradient driver system includes Gₓ (x direction), G_{y} (y direction), and G_{z} (z direction) amplifiers, etc. Each of the Gₓ, G_{y}, and G_{z} gradient amplifiers excites a corresponding gradient coil in the gradient coil assembly 142, to generate a magnetic field gradient used to spatially encode an MR signal during an MRI scan. The gradient coil assembly 142 is disposed within the resonance assembly 140, the resonance assembly further includes a superconducting magnet having a superconducting coil 144, and during operation, the superconducting coil provides a static uniform longitudinal magnetic field B₀ that runs through a cylindrical imaging volume 146. The resonance assembly 140 further includes an RF body coil 148, which, during operation, provides a transverse magnetic field B₁, and the transverse magnetic field B₁ is substantially perpendicular to B₀ throughout the entire cylindrical imaging volume 146. The resonance assembly 140 may further include an RF surface coil 149, and the RF surface coil is used to image different anatomical structures of the patient undergoing the MRI scan. The RF body coil 148 and the RF surface coil 149 may be configured to operate in a transmit and receive mode, a transmit mode, or a receive mode.

The x direction may also be referred to as a frequency encoding direction or a kₓ direction in k-space. The y direction may be referred to as a phase encoding direction or a k_{y} direction in the k-space. can be used for frequency encoding or signal readout, and is generally referred to as a frequency encoding gradient or a readout gradient. can be used for phase encoding, and is generally referred to as a phase encoding gradient. can be used for slice (layer) position selection to obtain k-space data. It should be noted that a layer selection direction, a phase encoding direction, and a frequency encoding direction may be modified according to actual requirements.

The subject or patient 170 of the MRI scan may be positioned within the cylindrical imaging volume 146 of the resonance assembly 140. The transceiver 135 in the MRI system controller 130 generates RF excitation pulses that are amplified by an RF amplifier 162 and provided to the RF body coil 148 by means of a transmit/receive switch (T/R switch) 164.

As described above, the RF body coil 148 and the RF surface coil 149 may be used to transmit an RF excitation pulse and/or receive obtained MR signals from the patient undergoing the MRI scan. MR signals emitted by excited nuclei in the patient of the MRI scan may be sensed and received by the RF body coil 148 or the RF surface coil 149 and sent back to a pre-amplifier 166 by means of the T/R switch 164. The T/R switch 164 may be controlled by a signal from the sequential pulse generator 133 to electrically connect, when in the transmit mode, the RF amplifier 162 to the RF body coil 148, and to connect, when in the receive mode, the pre-amplifier 166 to the RF body coil 148. The T/R switch 164 may further enable the RF surface coil 149 to be used in the transmit mode or the receive mode.

In some embodiments, the MR signals sensed and received by the RF body coil 148 or the RF surface coil 149 and amplified by the pre-amplifier 166 are stored as a raw k-space data array in the memory 137 for post-processing. A reconstructed magnetic resonance image may be obtained by transforming/processing the stored raw k-space data.

In some embodiments, the MR signals sensed and received by the RF body coil 148 or the RF surface coil 149 and amplified by the pre-amplifier 166 are demodulated, filtered and digitized in a receiving portion of the transceiver 135, and transmitted to the memory 137 in the MRI system controller 130. For each image that is to be reconstructed, the data is rearranged into separate k-space data arrays, each of the separate k-space data arrays is input to the array processor 139, and the array processor is operated to transform the data into an array of reconstructed image by Fourier transform.

The array processor 139 uses a transform method, most commonly Fourier transform, to create images from the received MR signals. These images are transmitted to the computer system 120 and stored in the memory 126. In response to commands received from the operator workstation 110, data for the reconstructed image may be stored in a long-term memory, or may be further processed by the image processor 128 and transmitted to the operator workstation 110 for presentation on the display 118.

In various embodiments, components of the computer system 120 and the MRI system controller 130 may be implemented on the same computer system or on a plurality of computer systems. It should be understood that the MRI system 100 shown in FIG. 1 is intended for illustration. Suitable MRI systems may include more, fewer, and/or different components.

The MRI system controller 130 and the image processor 128 may separately or collectively include a computer processor and a storage medium. The storage medium records a predetermined data processing program that is to be executed by the computer processor. For example, the storage medium may store a program used to implement scanning (for example, a scan procedure and an imaging sequence), image reconstruction, image processing, etc. For example, the storage medium may store a program used to implement the magnetic resonance imaging method according to the examples of the present invention. The above storage medium may include, for example, a ROM, a floppy disk, a hard disk, an optical disk, a magneto-optical disk, a CD-ROM, or a non-volatile memory card.

The aforementioned “imaging sequence” (also referred to below as a scan sequence or a pulse sequence) is a combination of pulses that have specific amplitudes, widths, directions, and time sequences, and that are applied when a magnetic resonance imaging scan is performed. These pulses typically may include, for example, a radio-frequency pulse and a gradient pulse. The radio-frequency pulses may include, for example, radio-frequency excitation pulses, radio-frequency refocusing pulses, inverse recovery pulses, etc. The gradient pulses may include, for example, the aforementioned gradient pulse used for layer selection, gradient pulse used for phase encoding, gradient pulse used for frequency encoding, gradient pulse used for phase shifting (phase shift), gradient pulse used for dispersion of phases (dephasing), etc.

Typically, a plurality of scan sequences can be preset in the magnetic resonance system, so that the sequence suitable for clinical detection requirements can be selected. The clinical detection requirements may include, for example, an imaging site, an imaging function, an imaging effect, and the like.

Currently, in a medical imaging system (such as a magnetic resonance imaging system or a computed tomography imaging system), an image capture apparatus may be introduced to obtain visual and morphological information of a human body. Typically, the image capture apparatus may be mounted near an examination bed to maximally acquire auxiliary information of a subject in a non-contact manner. In related technologies, a method of estimating body shape information of a subject based on a 3D image obtained by an image capture apparatus has been proposed.

In examples of the present application, a subject body index estimation method for medical imaging is proposed, including: extracting a first feature vector and key point information at a predetermined position of the body of the subject from image data captured by an image capture apparatus; generating a second feature vector based on the key point information; and estimating at least one of the height and the weight of the subject by combining the first feature vector and the second feature vector. Therefore, the height and weight of a human body can be estimated by combining a global first feature in original image data and a second feature related to human body morphology, that is, the height and weight of a human body can be estimated using multi-dimensional features, so that the estimation result is more accurate.

Description is made below in conjunction with the examples.

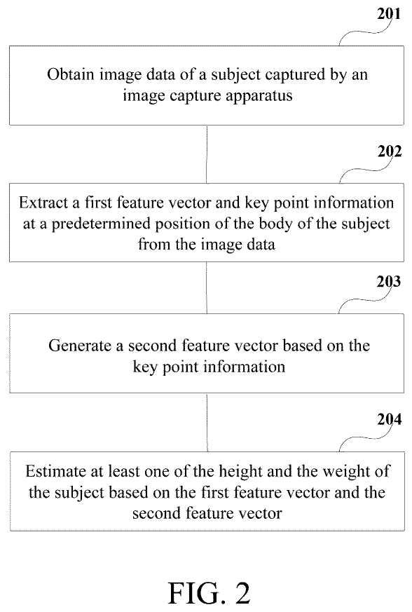

Examples of the present application provide a subject body index estimation method for medical imaging. FIG. 2 is a schematic diagram of a subject body index estimation method for medical imaging according to an example of the present application. As shown in FIG. 2, the method includes: at step 201, obtaining image data of a subject captured by an image capture apparatus.

At step 202, the method includes extracting a first feature vector and key point information at a predetermined position of the body of the subject from the image data.

The method further includes at step 203, generating a second feature vector based on the key point information; and at step 204, estimating at least one of the height and the weight of the subject based on the first feature vector and the second feature vector.

In some examples, the image data of the subject may be obtained by an image capture apparatus. The image data may include, but is not limited to, two-dimensional optical image data and depth image data. For example, the image capture apparatus may be a 3D camera. The 3D camera may capture the whole body of the subject in real time, to generate a video stream. Each frame in the video stream includes the two-dimensional optical image and the depth image. The two-dimensional optical image may be a two-dimensional RGB image. Each pixel value of the depth image reflects a distance between the camera and a corresponding position of the subject. The image capture apparatus may be mounted at a ceiling or a wall directly above an examination bed of a medical imaging system. Examples of the present application are not limited thereto.

In the above, the image capture apparatus being a 3D camera is used as an example, but the present application is not limited thereto. For example, at least one of the two-dimensional optical image data and the depth image data may also be obtained by an separate 2D optical camera and a depth sensor, respectively. No further examples will be provided herein.

In some examples, after the two-dimensional optical image data and the depth image data are obtained, the two-dimensional optical image data and the depth image data may be fused to generate image data. For example, the two-dimensional optical image data and the depth image data are fused to generate data of an RGBD structure as image data for ease of subsequent processing and analysis. The fusion processing includes registering the two-dimensional optical image data and the depth image data (for example, using a principle of perspective transformation), and the like, before synthesizing into data of an RGBD structure. For details, reference may be made to related technologies, which will not be repeated herein.

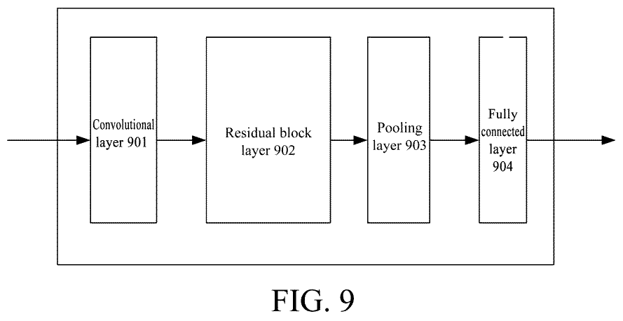

In some examples, in 202, the first feature vector may be extracted from the image data by using a convolutional neural network, including inputting the image data into the convolutional neural network, and using an output vector of the convolutional neural network as the first feature vector. FIG. 9 is a schematic diagram of a convolutional neural network according to an example of the present application. As shown in FIG. 9, the convolutional neural network includes at least a convolutional layer 901, a residual block layer 902, a pooling layer 903, and a fully connected layer 904, and each layer is composed of a plurality of processing nodes that may be referred to as neurons. Each neuron in each layer may perform a processing function and pass processed information to one neuron among a plurality of neurons in a downstream layer for further processing. For example, neurons in the first layer may learn to recognize structural edges in the image data. Neurons in the second layer may learn to recognize shapes based on the detected edges from the first layer, and so on and so forth. No further examples will be provided herein. For example, the convolutional neural network may use a ResNet model, a VGG model, a GoogleNet model, etc. Example of the present application are not limited thereto.

In some examples, the image data of a plurality of volunteers may be acquired in advance, the convolutional neural network may be trained by using the image data, and the trained convolutional neural network may be used to extract the first feature vector. The first feature vector may reflect (represent) at least one of the features such as an edge, texture, a shape, and a color in the image data. An output dimension of the convolutional neural network may be W1×1, and a value of W1 may be determined according to the convolutional neural network and actual requirements, for example, W1=4096, but examples of the present application are not limited thereto.

In some examples, in addition to extracting the first feature vector from the image data, the key point information at the predetermined position of the body of the subject may also be extracted from the image data. The key point information at the predetermined position may be extracted from the center of the image data by using a human body posture estimation model. The key point information may be coordinates in a camera coordinate system or coordinates in a medical imaging coordinate system, and the coordinates are three-dimensional coordinates in a space. That is, the image data is input into a pre-trained human body posture estimation model, and an output of the human body posture estimation model is coordinates of key points at a plurality of predetermined positions of a human body in a camera coordinate system or a medical imaging coordinate system. A coordinate origin of the medical imaging coordinate system may be a scanning central position (for example, an isocenter of a magnetic resonance imaging system), but examples of the present application are not limited thereto.

In some examples, the predetermined position may be a joint position having a certain degree of freedom on the human body, or an anatomical site of the human body easily identifiable in the image data, including, for example, at least two of a head top, a shoulder, a nose, an eye, an ear, an arm, an elbow, a wrist, a hip, a knee, and an ankle. For one predetermined position, if there are two symmetrical body positions on the left and right, then at least one of the two symmetrical body positions on the left and right may be included. For example, the predetermined position may include, but is not limited to, a left shoulder, a right shoulder, a left elbow, a right elbow, a left wrist, a right wrist, a left hip, a right hip, a left knee, a right knee, a left ankle, a right ankle, a left upper-arm, a right upper-arm, a left forearm, a right forearm, a left thigh, a right thigh, a left calf, a right calf, a head top, a head, a neck, a heart, a abdomen, and a pelvic cavity. The number of predetermined positions is not limited in examples of the present application.

In some examples, the human body posture estimation model is implemented based on a deep learning algorithm. For details, reference may be made to related technologies. For example, a densepose model, an openpose model, an HRNet model, and the like are used to extract the key point information from the image data. The image data of the plurality of volunteers, which is acquired in advance, may be used as an input parameter set, and pre-calibrated key point information at a plurality of predetermined positions corresponding to each piece of image data may be used as an output parameter set. The human body posture estimation model is trained by using the input parameter set and the output parameter set, and the trained human body posture estimation model is used to extract the key point information.

In some examples, the human body posture estimation model extracts, from the image data, coordinates (two-dimensional pixel coordinates) of a key point at a predetermined position in an image coordinate system. Camera parameters of an image capture apparatus may be determined. The camera parameters include intrinsic and extrinsic parameters of a camera. The intrinsic parameters include a focal point and a distance between a physical imaging plane and a optical center (i.e., a focal length f), and the extrinsic parameters include a rotation matrix, a scaling parameter, and a translation vector. The two-dimensional pixel coordinates may be converted into three-dimensional spatial coordinates of the camera coordinate system or three-dimensional spatial coordinates of the medical imaging coordinate system based on the camera parameters, to obtain the key point information. For coordinate conversion, reference may be made to related technologies, which will not be repeated herein.

In some examples, in 203, the second feature vector may be generated based on the key point information. As previously described, the key point reflects an anatomical structure of the human body. Therefore, a morphological feature may be determined based on a position of the anatomical structure as the second feature vector. The second feature vector may reflect (represent) a distance between anatomical structures of the human body, or a proportional relationship of distances. How to generate the second feature vector based on the key point information is described below.

In some examples, distances between at least two pairs (W2 pairs) of key points may be calculated based on the key point information; and the second feature vector may be generated based on the distances. An output dimension of the second feature vector may be W2×1, where W2 is greater than or equal to 2. W2 pairs of key points are selected from key points at predetermined positions, and each pair of key points includes two key points in a length direction or two key points in a width direction of the human body. A distance between each pair of key points is calculated, and the distance may be a distance in the length direction or a distance in the width direction of the human body, and may be obtained by calculating a difference between coordinates in the length direction or the width direction of each pair of key points. The W2 distances are distances with no overlapping area at all or distances with an overlapping area, and the present application is not limited thereto. The calculated W2 distances between W2 pairs of key points are arranged according to a predetermined rule to generate the second feature vector. According to the above example, since the height and weight of a human are correlated with the distances between the key points or the proportional relationship of the distances, the accuracy for estimating the height and weight can be improved based on the second feature vector.

In some examples, when each pair of key points includes two key points in the length direction of the human body, the two key points may be key points on the same side (the left side or right side) of the body, and when each pair of key points includes two key points in the width direction of the human body, the two key points may be left and right key points of the same anatomical site, but examples of the present application are not limited thereto.

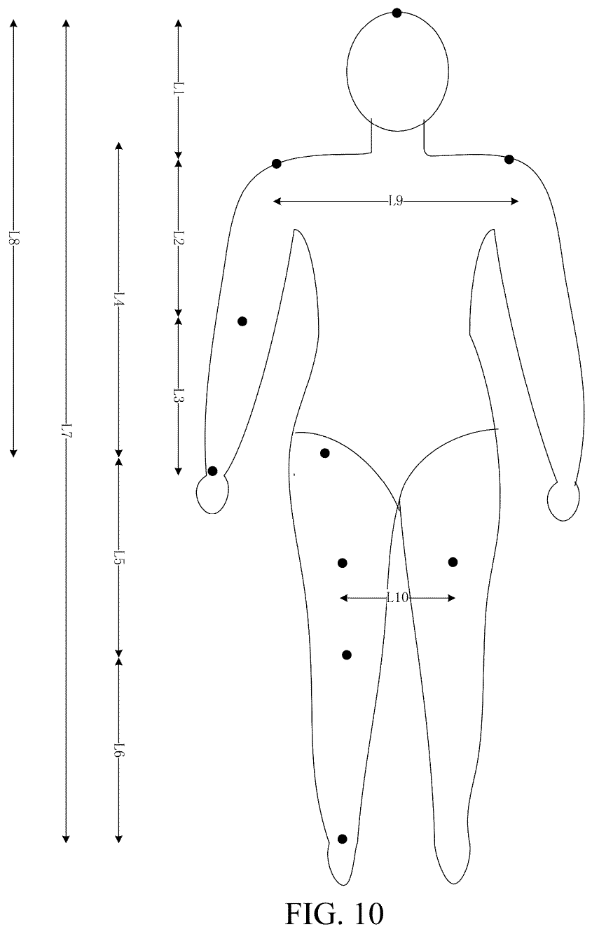

For example, for key points at the plurality of predetermined positions, W2=10 pairs of key points may be selected. FIG. 10 is a schematic diagram of key points at predetermined positions according to an example of the present application. As shown in FIG. 10, the 10 pairs of key points are respectively (head top, left shoulder), (left shoulder, left elbow), (left elbow, left wrist), (left shoulder, left hip), (left hip, left knee), (left knee, left ankle), (head top, ankle), (head top, left hip), (left shoulder, right shoulder), and (left thigh, right thigh). A distance L1 between the head top and the shoulder in the length direction is calculated based on the coordinates of the head top and the coordinates of the left shoulder. A distance L2 between the shoulder and the elbow in the length direction is calculated based on the coordinates of the left shoulder and the coordinates of the left elbow. A distance L3 between the elbow and the wrist in the length direction is calculated based on the coordinates of the left elbow and the coordinates of the left wrist. A distance L4 between the shoulder and the hip in the length direction is calculated based on the coordinates of the left shoulder and the coordinates of the left hip. A distance L5 between the hip and the knee in the length direction is calculated based on the coordinates of the left hip and the coordinates of the left knee. A distance L6 between the knee and the ankle in the length direction is calculated based on the coordinates of the left knee and the coordinates of the left ankle. A distance L7 between the head top and the ankle in the length direction is calculated based on the coordinates of the head top and the coordinates of the left ankle. A distance L8 between the head top and the hip in the length direction is calculated based on the coordinates of the head top and the coordinates of the left hip. A distance L9 between the left shoulder and the right shoulder in the width direction is calculated based on the coordinates of the left shoulder and the coordinates of the right shoulder. A distance L10 between the left thigh and the right thigh in the width direction is calculated based on the coordinates of the left thigh and the coordinates of the right thigh. The distances L1 to L3, L5, and L6 are separate distances that do not overlap each other. The distances L4, L7, and L8 are distances having overlapping areas with the distances L1 to L3, L5, and L6. The distances L1 to L8 are distances in the length direction. The distances L9 and L10 are distances in the width direction. The output dimension of the second feature vector may be W2×1, that is, after L1, L2, L3, L4, L5, L6, L7, L8, L9, and L10 are arranged, the generated vector (L1, L2, L3, L4, L5, L6, L7, L8, L9, L10)×1 is used as the second feature vector. The foregoing are only examples for description. Example of the present application are not limited thereto.

It should be noted that the foregoing distances refer to distances in the medical imaging coordinate system, that is, the distances reflect real distances between anatomical sites of the human body. When the key point information is three-dimensional spatial coordinates of the camera coordinate system, it is needed to perform coordinate conversion in combination with the camera parameters, before calculating a corresponding distance in the medical imaging coordinate system. For details, reference may be made to related technologies, which will not be repeated herein.

In some examples, in 204, at least one of the height and the weight of the subject may be estimated based on the first feature vector and the second feature vector, where at least one of the height and the weight of the subject may be estimated based on only the first feature vector and the second feature vector, or at least one of the height and the weight of the subject may be estimated based on the first feature vector, the second feature vector, and a third feature vector.

In some examples, in 204, an input feature vector may be generated based on concatenating only the first feature vector and the second feature vector. Concatenating the first feature vector and the second feature vector includes cascading the first feature vector and the second feature vector. The cascading may be in such a manner that all first feature vectors are in the front and all second feature vectors are in the back, or all second feature vectors are in the front and all first feature vectors are in the back, or the first feature vectors and the second feature vectors are alternately arranged at intervals. Example of the present application are not limited thereto. For example, if the dimension of the first feature vector is W1×1, and the dimension of the second feature vector is W2×1, then the dimension of the input feature vector is (W1+W2)×1.

In some examples, at least one of the height and the weight of the subject may be estimated by using a deep learning algorithm or a machine learning algorithm. The input feature vector generated through concatenation may be input to one or two regressors to obtain at least one of the height and the weight of the subject.

In some examples, the input feature vector may be input to one regressor to estimate both the height and the weight of the subject, or the input feature vector may be input to a height regressor to estimate the height of the subject, or the input feature vector may be input to a weight regressor to estimate the weight of the subject, or the input feature vector may be input to a weight regressor and a height regressor to estimate the weight and the height of the subject, respectively. The present application is not limited thereto.

In some examples, the regressor may be a support vector-based regressor (SVR). To be specific, the regressor may be used to determine an intrinsic relationship of the input feature vector and fit various types of data in the input feature vector, to predict the height and the weight. For a principle of the SVR, reference may be made to related technologies, and examples of the present application are not limited thereto. In addition, the regressor in the present application is not limited to the SVR, for example, the regressor may also be a linear regressor and the like. No further examples will be provided herein. Alternatively, it is also possible not to use the regressor, but another deep learning algorithm, and examples of the present application are not limited thereto.

In some examples, the one or two regressors may be pre-trained. For example, for a height regressor, input feature vectors of the plurality of volunteers may be obtained by using the method described above, and calibrated heights of the volunteers are used as an output to train the height regressor; and for a weight regressor, input feature vectors of the plurality of volunteers may be obtained by using the method described above, and calibrated weights of the volunteers are used as an output to train the weight regressor.

In some examples, before data is input to the regressor, a standardization preprocessing may be performed on the data. For example, the input feature vectors may be input to the regressors after passing through a fully connected layer. In the case of two different regressors, the input feature vectors need to be input into fully connected layers corresponding to the regressors, respectively, before being input to the regressors.

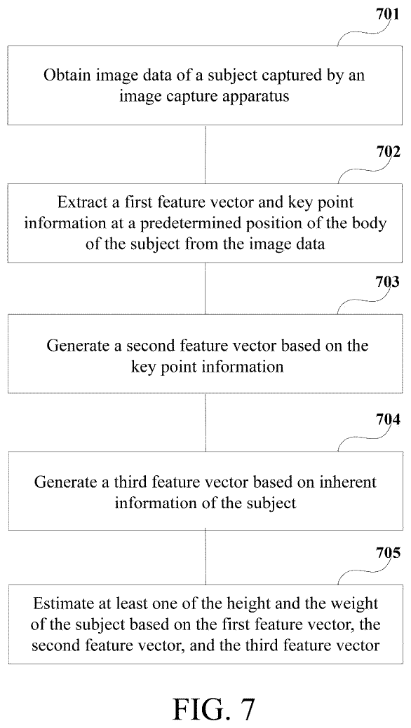

FIG. 7 is a schematic diagram of a subject body index estimation method for medical imaging according to an example of the present application. As shown in FIG. 7, the method includes: at step 701, obtaining image data of a subject captured by an image capture apparatus.

At step 702, the method includes extracting a first feature vector and key point information at a predetermined position of the body of the subject from the image data.

The method further includes at step 703, generating a second feature vector based on the key point information; and at step 704, generating a third feature vector based on inherent information of the subject. Finally at step 705, at least one of the height and the weight of the subject is estimated based on the first feature vector, the second feature vector, and the third feature vector.

In some examples, for the embodiments of 701 to 703, reference may be made to the foregoing embodiments, which will not be repeated herein. In 705, the difference from 204 is that the method of generating the input feature vector is different. To be specific, in 705, the input feature vector may be generated based on concatenating the first feature vector, the second feature vector, and the third feature vector, since the inherent information of the subject, in particular the gender and the age, is directly associated with the height and the weight of the subject. Therefore, in 704, the third feature vector may be generated based on the inherent information, including at least one of the age and the gender, of the subject. For example, the gender and the age of the subject may be extracted from a hospital database, and the gender and the age may be encoded to generate a third feature vector with a dimension of W3×1. A value of W3 is correlated with the number of categories of the inherent information. When only the gender or the age is extracted, W3=1, and when the gender and the age are extracted, W3=2. In addition, the inherent information may further include other types of human body attributes. Examples of the present application are not limited thereto. Concatenating the first feature vector with the second feature vector and the third feature vector includes cascading the first feature vector, the second feature vector, and the third feature vector (without limiting the sequence of the cascading). For example, if the dimension of the first feature vector is W1×1, the dimension of the second feature vector is W2×1, and the dimension of the third feature vector is W3×1, then the dimension of the input feature vector is (W1+W2+W3)×1.

It should be noted that FIG. 7 above merely schematically illustrates examples of the present application, but the present application is not limited thereto. For example, the order of execution between operations may be appropriately adjusted. In addition, some other operations may be added or some operations may be omitted. Those skilled in the art may make appropriate variations according to the above content, rather than being limited to the above disclosure of FIG. 7.

According to the foregoing example, the first feature vector and the key point information at the predetermined position of the body of the subject are extracted from the image data captured by the image capture apparatus; the second feature vector is generate based on the key point information; and at least one of the height and the weight of the subject is estimated by combining the first feature vector and the second feature vector. Therefore, the height and weight of a human body can be estimated by combining a global first feature in original image data and a second feature related to human body morphology, that is, the height and weight of a human body can be estimated using multi-dimensional features, so that the estimation result is more accurate.

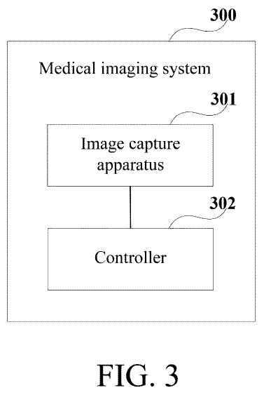

Examples of the present application further provide a medical imaging system. FIG. 3 is a schematic diagram of a medical imaging system according to an example of the present application. As shown in FIG. 3, the system 300 includes: an image capture apparatus 301, capturing image data of a subject; and a controller 302. The controller 302 is connected to the image capture apparatus 301, and configured to: receive the image data from the image capture apparatus; extract a first feature vector and key point information at a predetermined position of the body of the subject from the image data; generate a second feature vector based on the key point information; and estimate at least one of the height and the weight of the subject based on the first feature vector and the second feature vector.

Regarding the embodiments of the image capture apparatus 301 and the controller 302, reference may be made to the foregoing examples, which will not be repeated herein. The medical imaging system includes, but is not limited to, a computed tomography (CT) system, a magnetic resonance imaging (MRI) system, a C-arm imaging system, a positron emission computed tomography (PET) system, a single photon emission computed tomography (SPECT) system, an ultrasonic system, an X-ray imaging system, or any other suitable medical imaging system.

In some examples, the controller 302 may be configured separately from a controller of the medical imaging system. For example, the controller 302 is configured as a chip or the like connected to the controller of the medical imaging system, and the two controllers may control each other. Alternatively, functions of the controller 302 may also be integrated into the controller of the medical imaging system. Examples of the present application are not limited thereto.

In some examples, the controller 302 includes a computer processor and a storage medium. Recorded on the storage medium is a program for predetermined data processing to be executed by the computer processor. For example, stored on the storage medium may be a program for performing subject body index estimation for medical imaging. The storage medium may include, for example, a ROM, a floppy disk, a hard disk, an optical disk, a magneto-optical disk, a CD-ROM, or a non-volatile memory card.

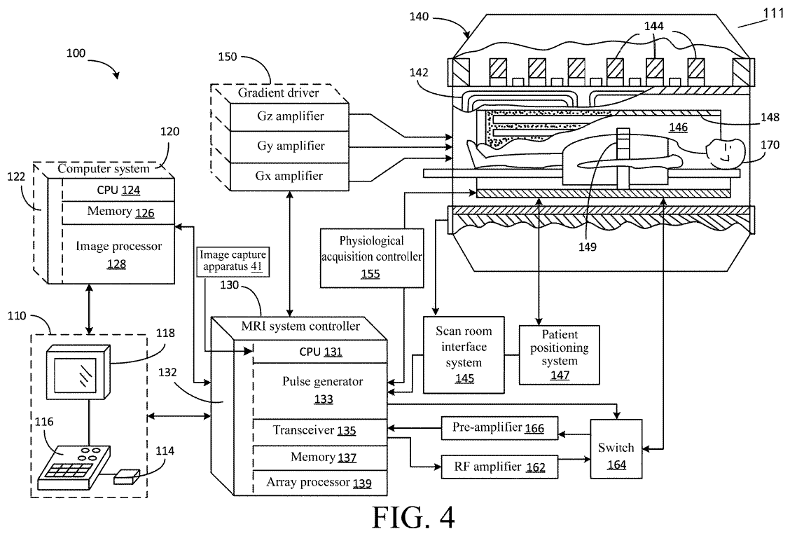

The medical imaging system may further include other structural components not shown in the figure. For details, reference may be made to related technologies, and examples of the present application are not limited thereto. Taking a magnetic resonance imaging system as an example, FIG. 4 is a schematic diagram of a magnetic resonance imaging system according to an example of the present application. As shown in FIG. 4, the difference from FIG. 1 is that the magnetic resonance imaging system further includes an image capture apparatus 41 that captures image data of a subject. For example, the image data includes two-dimensional optical image data and depth image data. In addition, a controller 130 or a computer system 120 of the magnetic resonance imaging system is connected to the image capture apparatus 41 and is configured to: receive the image data from the image capture apparatus; extract a first feature vector and key point information at a predetermined position of the body of the subject from the image data; generate a second feature vector based on the key point information; and estimate at least one of the height and the weight of the subject based on the first feature vector and the second feature vector. For a specific embodiment of the controller or the computer system, reference may be made to the foregoing examples, which will not be repeated herein.

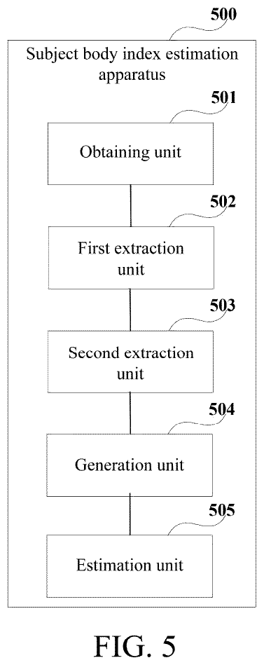

Examples of the present application further provide a subject body index estimation apparatus for medical imaging. FIG. 5 is a schematic diagram of a subject body index estimation apparatus for medical imaging according to an example of the present application. As shown in FIG. 5, the apparatus 500 includes: an obtaining unit 501, obtaining image data of a subject captured by an image capture apparatus; and a first extraction unit 502, extracting a first feature vector from the image data. The apparatus 500 also includes a second extraction unit 503, extracting key point information at a predetermined position of the body of the subject from the image data. The apparatus 500 further includes a generation unit 504, generating a second feature vector based on the key point information; and an estimation unit 505, estimating at least one of the height and the weight of the subject based on the first feature vector and the second feature vector.

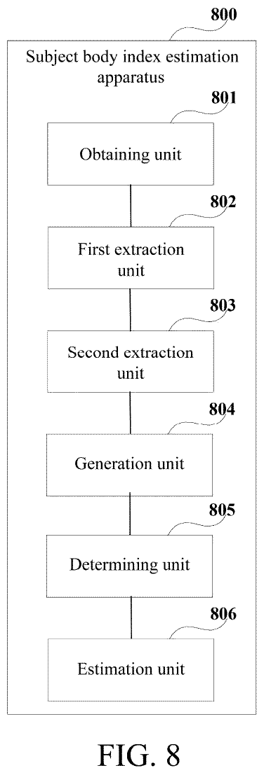

FIG. 8 is a schematic diagram of a subject body index estimation apparatus for medical imaging according to an example of the present application. As shown in FIG. 8, the apparatus 800 includes: an obtaining unit 801, obtaining image data of a subject captured by an image capture apparatus; and a first extraction unit 802, extracting a first feature vector from the image data. The apparatus 800 also includes a second extraction unit 803, extracting key point information at a predetermined position of the body of the subject from the image data; and a generation unit 804, generating a second feature vector based on the key point information. The apparatus 800 further includes a determining unit 805, generating a third feature vector based on inherent information of the subject; and an estimation unit 806, estimating at least one of the height and the weight of the subject based on the first feature vector, the second feature vector, and the third feature vector.

For the embodiments of modules of the foregoing units, reference may be made to 201 to 204, and the repeated description will not be provided again.

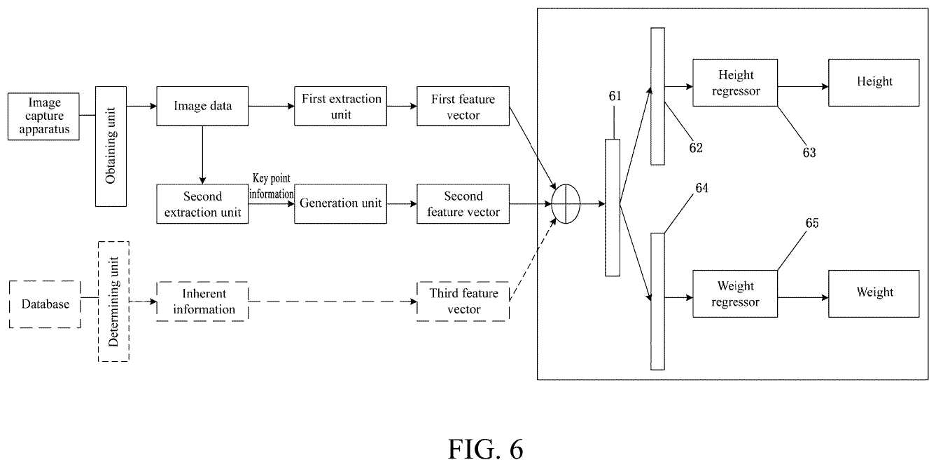

The following describes, in connection with FIG. 6, a procedure in which modules of the foregoing units estimate body indexes of the subject.

As shown in FIG. 6, an image capture apparatus captures image data of a subject, and the obtaining unit 501 or 801 obtains the image data. The image data is separately input to the first extraction unit 502 or 802 and the second extraction unit 503 or 803. The first extraction unit 502 or 802 extracts a first feature vector by using, for example, a convolutional neural network. The second extraction unit 503 or 803 extracts key point information by using a human body posture estimation model. The generation unit 504 or 804 generates a second feature vector based on the key point information. The determining unit 805 may further obtain inherent information of the subject from a database, and generate a third feature vector based on the inherent information. The estimation unit 505 generates an input feature vector by cascading the first feature vector and the second feature vector, or the estimation unit 806 generates an input feature vector by cascading the first feature vector, the second feature vector, and the third feature vector. The input feature vector is sequentially passed through a fully connected layer 61, a fully connected layer 62, and a height regressor 63, to predict the height, and the input feature vector is sequentially passed through the fully connected layer 61, a fully connected layer 64, and a weight regressor 65, to predict the weight.

Examples of the present application further provide a computer-readable program. The program, when executed in an apparatus or a medical imaging system, causes a computer to perform, in the apparatus or the medical imaging system, the subject body index estimation method for medical imaging described in the foregoing examples.

Examples of the present application further provide a storage medium having a computer-readable program stored therein. The computer-readable program causes a computer to perform, in an apparatus or a medical imaging system, the subject body index estimation method for medical imaging described in the foregoing examples.

The above apparatus and method of the present application can be implemented by hardware, or can be implemented by hardware in combination with software. The present application relates to the foregoing type of computer-readable program. When executed by a logic component, the program causes the logic component to implement the foregoing apparatus or a constituent component, or causes the logic component to implement various methods or steps as described above. The present application further relates to a storage medium for storing the above program, such as a hard disk, a magnetic disk, an optical disk, a DVD, a flash memory, etc.

The method/apparatus described with reference to the examples of the present application may be directly embodied as hardware, a software module executed by a processor, or a combination of the two. For example, one or more of the functional block diagrams and/or one or more combinations of the functional block diagrams as shown in the drawings may correspond to either software modules or hardware modules of a computer program flow. The foregoing software modules may respectively correspond to the steps shown in the figures. The foregoing hardware modules may be implemented, for example, by firming the foregoing software modules by using a field-programmable gate array (FPGA).

The software modules may be located in a RAM memory, a flash memory, a ROM memory, an EPROM memory, an EEPROM memory, a register, a hard disk, a removable disk, a CD-ROM, or any storage medium in other forms known in the art. The storage medium may be coupled to a processor, so that the processor can read information from the storage medium and can write information into the storage medium. Alternatively, the storage medium may be a constituent component of the processor. The processor and the storage medium may be located in an ASIC. The software module may be stored in a memory of a mobile terminal, and may also be stored in a memory card that can be inserted into a mobile terminal. For example, if a device (such as a mobile terminal) uses a large-capacity MEGA-SIM card or a large-capacity flash memory apparatus, then the software modules may be stored in the MEGA-SIM card or the large-capacity flash memory apparatus.

One or more of the functional blocks and/or one or more combinations of the functional blocks shown in the drawings may be implemented as a general-purpose processor, a digital signal processor (DSP), an application-specific integrated circuit (ASIC), a field-programmable gate array (FPGA) or other programmable logic devices, a discrete gate or transistor logic device, a discrete hardware assembly, or any appropriate combination thereof, which is used for implementing the functions described in the present application. The one or more functional blocks and/or the one or more combinations of the functional blocks shown in the drawings may also be implemented as a combination of computing equipment, such as a combination of a DSP and a microprocessor, a plurality of microprocessors, one or more microprocessors in communication combination with a DSP, or any other such configuration.

The present application is described above with reference to specific embodiments. However, it should be clear to those skilled in the art that the foregoing description is merely illustrative and is not intended to limit the scope of protection of the present application. Various variations and modifications may be made by those skilled in the art according to the principle of the present application, and said variations and modifications also fall within the scope of the present application.

1. A subject body index estimation method for medical imaging, characterized in that the method comprises:
obtaining image data of a subject captured by an image capture apparatus;
extracting a first feature vector and key point information at a predetermined position of the body of the subject from the image data;
generating a second feature vector based on the key point information; and
estimating at least one of the height and the weight of the subject based on the first feature vector and the second feature vector.

2. The method according to claim 1, wherein the first feature vector represents at least one feature among an edge, texture, a shape, and a color in the image data.

3. The method according to claim 1, wherein the second feature vector represents a distance between anatomical structures of the subject or reflects a proportional relationship of distances between anatomical structures of the subject.

4. The method according to claim 1, wherein the image data comprises two-dimensional optical image data and depth image data.

5. The method according to claim 1, characterized in that extracting the first feature vector and the key point information at the predetermined position of the body of the subject from the image data comprises:
extracting the first feature vector from the image data by using a convolutional neural network; and
extracting the key point information at the predetermined position of the body of the subject from the image data by using a human body posture estimation model.

6. The method according to claim 1, characterized in that the predetermined position comprises: at least two of a head top, a shoulder, a nose, an eye, an ear, an arm, an elbow, a wrist, a hip, a knee, and an ankle.

7. The method according to claim 1, characterized in that generating the second feature vector based on the key point information comprises:
calculating distances between at least two pairs of key points based on the key point information; and
generating the second feature vector based on the distances.

8. The method according to claim 7, characterized in that each pair of key points comprises two key points in the length direction or two key points in the width direction of a human body.

9. The method according to claim 1, characterized in that estimating at least one of the height and the weight of the subject based on the first feature vector and the second feature vector comprises:
concatenating the first feature vector and the second feature vector to generate an input feature vector; and
inputting the input feature vector into one or two regressors to obtain at least one of the height and the weight of the subject.

10. The method according to claim 1, characterized in that estimating at least one of the height and the weight of the subject based on the first feature vector and the second feature vector comprises:
generating a third feature vector based on inherent information of the subject;
concatenating the first feature vector, the second feature vector, and the third feature vector to generate an input feature vector; and
inputting the input feature vector into one or two regressors to obtain at least one of the height and the weight of the subject.

11. The method according to claim 10, characterized in that the inherent information comprises at least one of age and gender.

12. A computer-readable storage medium, comprising a stored computer program, wherein the subject body index estimation method for medical imaging according to claim 1 is performed when the computer program is run.

13. A medical imaging system, characterized in that the system comprises:
an image capture apparatus, capturing image data of a subject; and
a controller, connected to the image capture apparatus and used to perform the subject body index estimation method according to claim 1.
